(19) United States
(12) Patent Application Publication
(10) Pub. No.: US 2023/0141831 A1
(43) Pub. Date: May 11, 2023

(54) METHOD FOR MANAGING RESPIRATORY INFECTIONS THROUGH MOBILE TERMINAL MANAGEMENT SYSTEM INCLUDING CENTRAL MANAGEMENT SERVER, SERVER, AND COMPUTER READABLE STORAGE MEDIUM

(71) Applicant: SEEGENE, INC., Seoul (KR)

(72) Inventor: Jong Yoon CHUN, Gyeonggi-do (KR)

(21) Appl. No.: 17/913,298

(22) PCT Filed: Mar. 22, 2021
(86) PCT No.: PCT/KR2021/003525

§ 371 Date: Sep. 21, 2022

(30) Foreign Application Priority Data
Mar. 24, 2020 (KR) 10-2020-0035933
Mar. 30, 2020 (KR) 10-2020-0038634

(51) Int. Cl. G16H 10/65

(52) U.S. Cl. CPC G16H 10/65

(57) ABSTRACT

The present invention relates to a method for managing respiratory infections, a server, and a computer readable storage medium which conducts a respiratory infection test to a client who wants to the respiratory infection tests, receives the client's location information, and provides respiratory infection management information generated using the test result of the respiratory infection and client's location information for other clients so that it may minimize the spread of infection and efficiently quarantine against the respiratory infections while maintaining social and economic activities of the public.

### TECHNICAL FIELD

The present invention relates to a method for managing respiratory infections, a server, and a computer readable storage medium which conducts a respiratory infection test to a client who wants to the respiratory infection tests, receives the client's location information, and provides respiratory infection management information generated using the test result of the respiratory infection and client's location information for other clients so that it may minimize the spread of infection and efficiently quarantine against the respiratory infections while maintaining social and economic activities of the public.

### BACKGROUND ART

SARS CoV-2, a respiratory infection that has recently spread all over the world, may infect not only people with whom it has been contacted, but also family members, and if the infection is confirmed, it may be included in the control object that makes it difficult for individuals to live their daily lives. In addition, since SARS CoV-2 was not properly tested for respiratory infections in the early stages of the infection, the infection spread worldwide through various routes and proceeded as a pandemic.

In particular, the respiratory infections, including SARS CoV-2 and the like, cause serious mortality in children, the elderly and those with weakened immune systems, and those suffering from the underlying disease. Accordingly, it is very important to control the infection and identify causative pathogens for proper patient management. Most of the tests for the respiratory infections have developed rapidly with the development of molecular diagnostic technology, especially real-time polymerase chain reaction (PCR). The Real-time PCR is much more sensitive than traditional methods, and may detect a variety of pathogens quickly and simultaneously.

In order to use the molecular diagnostics for testing for the respiratory infections, specimens of suspected cases must be collected. However, due to the nature of the respiratory infections in which the infection is transmitted through the respiratory tract, the specimen collector who comes into contact with the suspected patient is seriously concerned about the infection from the suspected patient. Accordingly, the specimen collector wears protective clothing including a mask, etc. and collects the specimens from the suspected patients.

When a patient occurs due to the nature of respiratory infections, the number of suspected infected patients may rapidly increase because the transmission rate is very fast unlike other infectious diseases. Therefore, in order to perform a respiratory infection test from a rapidly increasing number of the suspected patients, all suspected patients must be sampled first. If a health professional included in a medical agency devotes time to sample collection, there may be a problem in that the treatment of a patient who is determined to be positive by the test of the respiratory infections may be delayed.

Therefore, if the collection of specimens in the medical agencies is avoided, and a method for collecting specimens by a suspected patient and a system capable of performing such a method are provided, quarantine against the respiratory infections may be performed more efficiently.

In addition, the suspected patients who have been tested positive for the respiratory infections should conduct an epidemiological investigation on the situation in which the infection may spread from after the infection occurs, but it is difficult to collect objective movement information of the suspected patient so that there may cause a problem that the quarantine is not easy.

Therefore, when the objective movement information is generated by collecting location information of the suspected patients, an easy epidemiological investigation may be performed, and the efficient quarantine may be achieved accordingly.

On the other hand, in order to the respiratory infection tests of the suspected patients with the respiratory infections, the suspected patient must visit the hospital directly, but there are cases where it is impossible for children with severe symptoms, the elderly, and severely disabled people with disabilities to visit a medical agency and collect specimens.

In addition, face-to-face contact is not possible due to a suspicion of the respiratory infection, and a suspected patient included in the quarantine may not be able to visit the medical agency, so it is not possible to collect the specimen for test of the suspected patient who has symptoms of the respiratory infection.

Therefore, even if it is not the medical agency or a specimen collection site that may replace the medical agency, a method for collecting the specimen from a place where the suspected patient wants to collect the specimen must be devised.

### DISCLOSURE OF INVENTION

### Technical Problem

The inventors have struggled to come up with a method for minimizing the spread of infection and efficient quarantine by using information generated from the client's mobile terminal in order to be able to actively maintain social and economic activities for a client who requests a respiratory infection test. As a result, the inventors have identified that the present invention provides the respiratory infection management information generated using the location information of the client actively requesting the respiratory infection test and the test result for the respiratory infection, thereby performing efficient quarantine through rapid treatment of the client and epidemiological investigation.

In addition, the present invention provides a sampling kit for specimen collection to a client who has requested the respiratory infection test, and the client provides the sampling kit in which the specimen is accommodated to a test agency, so that a medical agency may secure it without directly collecting the specimen from the client to test the client's respiratory infection more efficiently.

In addition, since the present invention may provide a sampling kit for securing a specimen to a client in various ways so that it may prevent unnecessary movement paths from occurring to a suspected client of the respiratory infection.

In addition, the present invention provides an application for a mobile terminal through which a user suspected of the respiratory infection may apply for a respiratory infection test through a personally owned mobile terminal, so that it may provide information for a more accurate respiratory infection test of a client for a government agency, the test agency, the treatment agency and the like. It has been confirmed that duplicate creation and security of client information may be maintained by allowing it to be used in each process of tests.

Accordingly, the present invention aims to provide a method for managing the respiratory infections through a mobile terminal management system including a central management server.

In addition, the present invention also aims to provide a mobile terminal management system for managing the respiratory infections.

In addition, the present invention also aims to provide a computer-readable storage medium for managing the respiratory infections.

In addition, the present inventors have struggled to come up with a method, a server, etc. for collecting specimens of severely ill patients and elderly people with disabilities. As a result, the inventors have identified that the present invention provides a sampling kit for specimen collection to a user, and provides the sampling kit in which the client's specimen is accommodated to a test agency, so that a medical agency may secure it without directly collecting the specimen from the client to test the client's respiratory infection more efficiently.

In addition, In addition, since the present invention may provide a sampling kit for securing a specimen to a client in various ways so that it may prevent unnecessary movement paths from occurring to a suspected client of the respiratory infection.

Accordingly, the present invention aims to provide a method for testing a disease through remote specimen sampling.

In addition, the present invention also aims to provide a server for disease testing through the remote specimen sampling.

In addition, the present invention also aims to provide a computer-readable storage medium for disease testing through remote specimen sampling.

The other objectives and advantages of the present invention will be apparent to the embodiments and claims described below in detail with reference to the accompanying drawings.

### Solution to Problem

According to an embodiment of the present invention, there is provided a method comprising the following steps of managing respiratory infections through a mobile terminal management system comprising a central management server. The following steps of managing respiratory infections includes; receiving, by the central management server, a request for a respiratory infection test from a client's mobile terminal; receiving, by the central management server, location information from the mobile terminal to generate movement information; requesting, by the central management server, the respiratory infection test for the client to a terminal of a test agency; receiving, by the central management server, a test result of the respiratory infection of the client from the terminal of the test agency; and generating, by the central management server, the respiratory infection management information using the movement information and the test result.

According to an embodiment of the present invention, there is provided a central management server for managing respiratory infections using a memory, a processor, and one or more programs stored in the memory and configured to be executed by the processor, wherein when the one or more programs are executed by one or more processors, the one or more programs comprises instructions to perform: receiving, by the central management server, a request for a respiratory infection test from a client's mobile terminal; receiving, by the central management server, location information from the mobile terminal to generate movement information; requesting, by the central management server, the respiratory infection test for the client to a terminal of a test agency; receiving, by the central management server, a test result of the respiratory infection of the client from the terminal of the test agency; and generating, by the central management server, the respiratory infection management information using the movement information and the test result.

According to an embodiment of the present invention, there is provided a non-transitory computer-readable storage medium that stores an instruction which performs a method for managing respiratory infections by one or more processors in a computing device when executed by the one or more processors, wherein when the instruction is executed by the one or more processors, the instruction enables the computing device to perform a method comprising the following step: receiving, by the central management server, a request for a respiratory infection test from the client's mobile terminal; receiving, by the central management server, location information from the mobile terminal to generate movement information; requesting, by the central management server, the respiratory infection test for the client to a terminal of a test agency; receiving, by the central management server, a test result of the respiratory infection of the client from the terminal of the test agency; and generating, by the central management server, the respiratory infection management information using the movement information and the test result.

According to an embodiment of the present invention, there is provided a terminal connected to the above-described central management server of claim 1 through a communication network and installed with an application that performs the function of a mobile terminal in each step of performing a method of managing respiratory infections.

According to an embodiment of the present invention, there is provided a disease testing method through remote specimen sampling, the disease testing method comprising: receiving, by a server, disease test request information provided from a user's mobile terminal wherein the server selects a sampling kit for a disease test corresponding to the disease test request information; providing, by the server, instruction information to a delivery agency terminal providing the selected sampling kit wherein the instruction information comprises information on a sampling kit for the disease test and delivery information; receiving, by the server, identifier information from the mobile terminal and authenticating the user using the delivered sampling kit wherein the identifier information is generated by the mobile terminal recognizing an identifier comprised in the sampling kit, and the server checks the identifier information to authenticate the user; authenticating, by the server, the collected sampling kit by receiving identifier information from a terminal of a test agency wherein the test agency is an agency in which a sampling kit containing the user's specimen is collected, and the identifier information is generated by the terminal of the test agency recognizing an identifier comprised in the sampling kit collected from the user, and the server checks the received identification information to authenticate the sampling kit; receiving, by the server, a test result from the test agency terminal wherein the test result is generated by a test apparatus performing the disease test on the specimen comprised in the sampling kit; and providing, by the server, the received test result to the mobile terminal.

According to an embodiment of the present invention, there is provided a central management server for performing a disease testing through remote specimen sampling using a memory, a processor, and one or more programs stored in the memory and configured to be executed by the processor, wherein when the one or more programs are executed by one or more processors, the one or more programs comprises instructions to perform: receiving, by the central management server, disease test request information provided from the user's mobile terminal wherein the server selects a sampling kit for a disease test corresponding to the disease test request information; providing, by the central management server, instruction information to a delivery agency terminal providing the selected sampling kit wherein the instruction information comprises information on a sampling kit for the disease test and delivery information; receiving, by the central management server, identifier information from the mobile terminal and authenticating the user using the delivered sampling kit wherein the identifier information is generated by the mobile terminal recognizing an identifier comprised in the sampling kit, and the server checks the identifier information to authenticate the user; authenticating, by the central management server, the collected sampling kit by receiving identifier information from a terminal of a test agency wherein the test agency is an agency in which a sampling kit containing the user's specimen is collected, and the identifier information is generated by the terminal of the test agency recognizing an identifier comprised in the sampling kit collected from the user, and the server checks the received identification information to authenticate the sampling kit; receiving, by the central management server, a test result from the test agency terminal wherein the test result is generated by a test apparatus performing the disease test on the specimen comprised in the sampling kit; and providing the received test result to the mobile terminal.

According to an embodiment of the present invention, there is provided a non-transitory computer-readable storage medium that stores an instruction which performs a method for managing respiratory infections by one or more processors in a computing device when executed by the one or more processors, wherein when the instruction is executed by the one or more processors, the instruction enables the computing device to perform: receiving disease test request information provided from the user's mobile terminal wherein the server selects a sampling kit for a disease test corresponding to the disease test request information; providing instruction information to a delivery agency terminal providing the selected sampling kit wherein the instruction information comprises information on a sampling kit for the disease test and delivery information; receiving identifier information from the mobile terminal and authenticating the user using the delivered sampling kit wherein the identifier information is generated by the mobile terminal recognizing an identifier comprised in the sampling kit, and the server checks the identifier information to authenticate the user; authenticating the collected sampling kit by receiving identifier information from a terminal of a test agency wherein the test agency is an agency in which a sampling kit containing the user's specimen is collected, and the identifier information is generated by the terminal of the test agency recognizing an identifier comprised in the sampling kit collected from the user, and the server checks the received identification information to authenticate the sampling kit; receiving a test result from the test agency terminal wherein the test result is generated by a test apparatus performing the disease test on the specimen comprised in the sampling kit; and providing the received test result to the mobile terminal.

According to an embodiment of the present invention, there is provided a terminal connected to the above-described server of claim 13 through a communication network and installed with an application that performs the function of a mobile terminal in each step of performing a disease testing method through remote specimen sampling.

### Advantageous Effects of Invention

The features and advantages of the present invention may be summarized as follows:

(1) According to the present invention, the method, system, and storage medium the method, the system, the central management server, and the computer-readable storage medium may minimize the spread of infection while maintaining the social and the economic activities of the people, and efficiently quarantining.

(2) According to the present invention, the method, the system, the central management server, and the computer-readable storage medium enables rapid response at the national level in the event of an outbreak of an infectious virus, thereby preventing the early spread of the respiratory infections.

(3) According to the present invention, the method, the system, the central management server, and the computer-readable storage medium may promptly test a respiratory infection through an active test application from a suspected client of the respiratory infection and continuously receive the client's location information in real-time, thereby generating respiratory infection management information using accumulated test results and location information of each client.

(4) According to the present invention, the method, the system, the central management server, and the computer-readable storage medium may provide the test application of the respiratory infection and location information by installing an application that allows the suspected clients of the respiratory infection to access the mobile terminal management system on their own personal mobile terminals, so that all information generated when the respiratory infection occurs may be not exposed to the outside and be managed and stored in the central management server.

(5) According to the present invention, the method, the system, the central management server, and the computer-readable storage medium may provide a mobile terminal management system that may deliver and retrieve a sampling kit that may sample specimens to the client's location even if the suspected client of the respiratory infection is quarantined at home or located in an area that cannot be tested for the respiratory infection, thereby quickly preventing the spread of infection through early detection and early treatment of infectious diseases.

(6) According to the present invention, the method, the system, the central management server, and the computer-readable storage medium may provide an infection map including information on the location and the status of the clients (negative/positive status, test date, etc.), thereby identifying hazardous areas (areas requiring quarantine) and non-hazardous areas, setting individual movements, and adjusting the scope of activities.

(7) According to the present invention, the method, the system, the central management server, and the computer-readable storage medium may deliver a self-sampling kit within 1 to 2 hours after the client's test request, and promptly receive the test result for the respiratory infection within 24 hours.

(8) According to the present invention, the method, the system, the central management server, and the computer-readable storage medium may quickly resolving the client's movement restriction state (for example, quarantine during entrance procedures, quarantine after visiting a hazardous area, quarantine after contact with an infected person, etc.) according to the client's rapid test of the suspected respiratory infection.

(9) According to the present invention, the method, the system, the central management server, and the computer-readable storage medium may standardize the level of quarantine because the same platform is used in regions or countries following the platform of the same mobile terminal management system, thereby reducing distrust between regions or countries due to differences in quarantine levels.

(10) According to the present invention, the method, the system, the central management server, and the computer-readable storage medium may provide compatibility, interoperability, safety, repeatability, and excellent quality of the mobile terminal management system according to using the standardized platform.

(11) According to the method, the system, the central management server, and the computer-readable storage medium of the present invention, the flow, the processing and the storage of information can be securely and integrated through the system and server established at the national level, thereby establishing a public infrastructure for early detection and early treatment of infectious diseases.

(12) According to the method, the system, the central management server, and the computer-readable storage medium of the present invention, as the accuracy and usefulness of information derived from data accumulated according to increase of installation of applications accessible to the mobile terminal management system on each mobile terminal of individuals or requests for infectious disease test from clients is increased, so unnecessary quarantine or control may be minimized through epidemiological conclusions generated using this.

(13) According to the present invention, the method, the system, the central management server, and the computer-readable storage medium may generate the predicted contact information using the location information of the suspected client of the respiratory infection, and request the respiratory infection tests to other users according to the generated predicted contact information.

(14) According to the present invention, the method, the system, the central management server, and the computer-readable storage medium, may generate the predicted contact information using the location information of the suspected client of the respiratory infection and reflect the generated predicted contact information in the calculation of reproduction number, R), thereby easily establishing of a quarantine plan for the respiratory infections.

(15) According to the present invention, the method, the server, and the computer-readable storage medium may provide the sampling kit for specimen collection to the elderly, the severely symptomatic, or the suspected infectious disease with limited mobility, and the like, thereby carrying out the disease test process without visiting a medical agency.

(16) According to the present invention, the method, the server, and the computer-readable storage medium may omit visit to the medical agency for specimen collection when the suspected disease is an infectious disease, thereby reducing the infection rate of medical workers.

(17) According to the present invention, the method, the server, and the computer-readable storage medium may lower the cost of the test since no specimen collection action of a medical worker by visiting the medical agency occurs.

(18) According to the present invention, the method, the server, and the computer-readable storage medium may provide convenient access to the test to a user showing a symptom suspected of a disease, thereby providing a medical service for many users.

(19) According to the present invention, the method, the server, and the computer-readable storage medium may perform user authentication, authentication and the like of a sampling kit using an application installed in a smartphone carried by an individual and request the disease test, thereby preventing personal medical information from being exposed to the outside.

(20) According to the method, the server, and the computer-readable storage medium of the present invention, users who are quarantined at home may also actively participate in the test, and test result data according to this may be transmitted to national agencies, thereby being able to share information on prevention and response of the infectious disease with the world in real time.

(21) According to the present invention, the method, the server, and the computer-readable storage medium may, the server may provide a video manual of the sampling kit that is used differently depending on the disease with the user's smartphone, so that the user may more conveniently collect specimens.

(22) According to the present invention, the method, the server, and the computer-readable storage medium may, the server may provide a checklist to be noted in the process of taking the specimen using the sampling kit with a user's smartphone, thereby being able to clearly identify the rules to be done when collecting the specimen.

### BRIEF DESCRIPTION OF DRAWINGS

FIG. 1 is a view schematically illustrating a configuration of a mobile terminal management system including a central management server according to a first aspect of the present invention;

FIG. 2 is a flowchart for illustrating a method of managing a respiratory infection performed using the mobile terminal management system according to the first aspect of the present invention;

FIG. 3 is an exemplary view illustrating a sampling kit according to an embodiment of the present invention;

FIG. 4 is an exemplary view for illustrating an infection map according to an embodiment of the present invention;

FIG. 5 is a view schematically illustrating a configuration of a disease test system through remote specimen sampling according to a second aspect of the present invention;

FIG. 6 is a view schematically illustrating a configuration of a disease test system through the remote specimen sampling according to an embodiment of the second aspect of the present invention; and

FIG. 7 is a flowchart illustrating a method for testing a disease through the remote specimen sampling according to the second aspect of the present invention.

### MODE FOR THE INVENTION

The configuration and effects of the present invention are now described in further detail in connection with embodiments thereof. The embodiments are provided merely to specifically describe the present invention, and it is obvious to one of ordinary skill in the art that the scope of the present invention is not limited to the embodiments.

In addition, in describing the component elements of the present invention, terms such as first, second, A, B, (a), (b), (i), and (ii) may be used. These terms are only for distinguishing the component from other components, and the nature, turn, or order of the component is not limited by the term. If a component is described as being “connected” or “coupled” to another component, the component may be directly connected to another component, but it should be understood that another component between each component may be “connected” or “coupled”.

The term “client” or “user” used in the present invention is a person who has a personal mobile terminal (hereinafter, “mobile terminal”). The client or user may be a suspected patient with expected respiratory infection. The client or user may be a public person for whom a respiratory infection is not expected. The client or user may be a user who has been cured after the respiratory infection has occurred. The client or user may request a respiratory infection test using the mobile terminal. The client or user may provide location information on the current location to the central management server using the mobile terminal. The client or user may provide the test result of the respiratory infection to the central management server. The client or user may receive information transmitted from the central management server using the mobile terminal. The client or user can check the infection map transmitted from the central management server through the owned mobile terminal.

The term “test agency” used in the present invention may be a large hospital or a medical test center (C-Lab) that has a test center. The test agency is an agency that may quickly test and report the results of specimens sampled from the clients. At least one test agency may be selected, and has a test agency terminal capable of communicating with the central management server. The test agency may provide sampling kits to the clients requesting tests for the respiratory infections. The test agency has testing equipment capable of performing the respiratory infection tests on sampled clients' specimens. The test agency may directly ship the sampling kits provided to the client, or they may provide them through a delivery agency of choice. The test agency may retrieve the sampling kit from which the client's specimen was collected. The test agency may directly collect the sampling kit containing the client's specimen or collect it through the selected recovery agency. In this case, the delivery agency and the collection agency may be the same or not the same agency. The test agency ships and retrieves the sampling kit to the client's desired location. If necessary, the test agency may retrieve the sampling kit at a location desired by the client rather than at the location where it was delivered.

The term “treatment agency” used in the present invention is a hospital that may manage patients designated by a national agency. The treatment agency is a medical agency that may manage patients, such as diagnosis and treatment for clients (patients) who have been tested positive for the respiratory infections (eg, SARS CoV-2). The treatment agency may receive or provide information from/to the central management server through a communication network. At least one treatment agency may be selected and has a treatment agency terminal capable of communicating with the central management server. The treatment agency includes a transport means capable of transporting a positively determined client (patient). When the treatment agency receives the information of the positive client from the central management server, the positive client may be transferred from the location of the positive client to the treatment agency using the transport means.

The term “central management server” used in the present invention is managed by the national agency. The central management server may be managed by private agency or private enterprises delegated by the national agency. The central management server may provide and receive information through the communication network such as mobile terminals of a plurality of the clients, terminals of a plurality of the test agencies, terminals of a plurality of the treatment agencies, and the like. The creation, the provision, the storage, and the reception of information performed by the central management server may be performed automatically by the central management server, or may be performed manually under the control of the central management server by a person in charge of the national agency (or the private agency, the private enterprise). The national agency, the private agency, or the private enterprise that manage the central management server include the transport means to transport the positively determined clients (patients).

The term “national agency” used in the present invention is an agency that is directly affiliated with a government organization, and is an organization capable of managing and controlling a mobile terminal management system. The national agency is, for example, an agency under the Ministry of Health and Welfare or Centers for Disease Control and Prevention. The national agency may control the central management server. The national agency may directly control the central management server, and, if necessary, may control the central management server through the national agency terminal used by the national agency.

The term “private agency” or “private enterprise” used in the present invention refers to an agency or an enterprise that may perform management and control a mobile terminal management system according to the present invention by being delegated by the national agency. The private agency or the private enterprise may directly control the central management server, and, if necessary, control the central management server through the private agency terminal or the private enterprise terminal used in the private agency or the private enterprise. The private agency or the private enterprise may provide information processed by the mobile terminal management systems to the national agency.

The term ‘mobile terminal’ used in the present invention is a portable communication device (for example, a mobile phone, a smartphone, etc.) possessed by the client, and is connected to the central management server of the mobile terminal management system through the communication network to provide and receive information to/from it. The mobile terminal is equipped with an application capable of providing and receiving information to/from the central management server. The mobile terminal may collect location information (eg, location information by a GPS signal or location information by a communication base station). The location information may include time information. Each client may own the mobile terminal. The personal information of the client is stored in the mobile terminal. The mobile terminal includes one or more authentication means for verifying the client. The authentication means stored in the mobile terminal includes a public certificate, biometric authentication (iris, fingerprint, face, etc.), password authentication, and the like.

The term “application (or App.)” used in the present invention is a program installed in a client's mobile terminal. The application may be an accredited program that may be provided by the national agency. The application may provide information input by the client and/or information stored in the mobile terminal to the central management server. The application may provide the location information collected from the mobile terminal to the central management server. The application may provide the location information to the central management server in real time, continuously and/or periodically. The application may receive and display information provided by the central management server. The application may input information provided to the central management server for the respiratory infection tests. The application may receive delivery/retrieve information of the sampling kit provided by the central management server, and the transport information transported the positively determined clients to the treatment agency. The application may receive and display an infection map provided by the central management server.

The term “sampling kit” used in the present invention is a container capable of holding the specimen collected from the client. The sampling kit includes a self-sampling kit. The sampling kit consists of one or more containers, and one configured container is a container for receiving a specimen. The sampling kit may include an additional container containing a material for sampling the specimen in addition to the container containing the specimen. The sampling kit may include protective equipment used to retrieve the specimen. The sampling kit is shipped and/or retrieved through the test agency (or consigned agency).

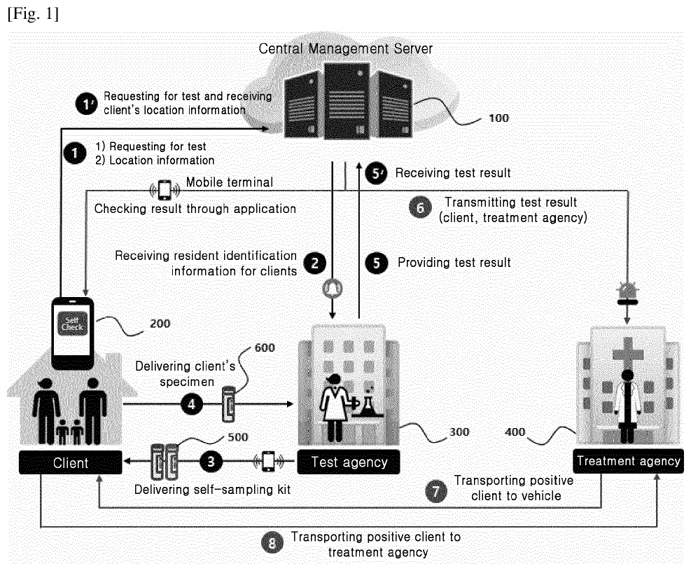

FIG. 1 is a view schematically illustrating a configuration of a mobile terminal management system including a central management server according to a first aspect of the present invention.

As shown in FIG. 1, the client(patient) suspected of the respiratory infection may actively request the respiratory infection test to the central management server 100 of the mobile terminal management system operated by the national agency through the application installed on the personal mobile terminal 200. The request for the respiratory infection test performed by the mobile terminal 200 is provided to the central management server 100 together with information input through the application and/or information stored in the mobile terminal 200. The mobile terminal 200 provides the location information of the mobile terminal 200 together with the request for the respiratory infection test to the central management server 100 through the application.

Information input through the application included in the request for the test of the respiratory infection may include information on the health status of the client (e.g., a questionnaire). The health status information may be created in a manner in which the central management server 100 responds to questions such as a questionnaire provided to the application. In addition, the health status information may be prepared in such a way that the client describes the current status of the client's health. The contents included in the health status information may include a variety of information that allows a medical worker to determine the client's condition, such as the client's body temperature, whether he has visited a risk area, whether he has contacted a person with the respiratory infection, or difficulty breathing. At this time, a basic questionnaire may also be included in the health status information.

The location information is information that may be generated by the mobile terminal 200 and is generated by receiving a GPS signal and a communication base station signal. The location information is continuously generated in real time, and the application provides the generated location information to the central management server 100 in real time, continuously and/or periodically. Accordingly, the location information of the client (carrying a mobile terminal) requesting the respiratory infection test is provided to the central management server 100.

The client may receive the delivery information of the sampling kit from the central management server according to the test request. The sampling kit capable of accommodating the client's specimen according to the client's active test request can be sent to the client immediately after the test request, and the delivery information for this may be checked through the client's mobile terminal.

The sampling kit may be provided by only one test agency. Alternatively, the sampling kit may be provided through the test agency selected from among a plurality of the test agencies. The sampling kit may be provided to clients using a variety of transport means.

The delivery period of the sampling kit according to an embodiment of the present invention may be within 1 to 2 hours of the test request. The delivery period of the sampling kit according to another embodiment of the present invention may be within 5 to 10 hours of the test request. The delivery period of the sampling kit according to another embodiment of the present invention may be within 1 day.

The delivered sampling kit may contain various components depending on the type of the respiratory infection. In one embodiment, the sampling of saliva and upper respiratory tract swabs (including separate instruments) may include only a specimen transport container for accommodating the specimen. In another embodiment, the sampling kit for oral sampling may include a container for accommodating a physiological saline solution for gargle and a container for accommodating a specimen.

The client receives the sampling kit delivered to the client's requested location according to the delivery information, and collects the specimen in the sampling kit. The client may perform a collection request through the application of the mobile terminal 200 so that the sampling kit accommodating the specimen is retrieved to the test agency. The central management server 100 receives the collection request and provides it to the test agency 300.

The client checks the collection information provided by the central management server 100 through the mobile terminal 200 and provides the sampling kit to the test agency 300. The client provides information notifying that the test agency 300 has retrieved the sampling kit through the mobile terminal 200 to the central management server 100 or the test agency terminal 300.

The mobile terminal 200 may receive respiratory infection management information such as an infection map provided by the central management server 100. The generated respiratory infection management information may include the test result of the client who requested the respiratory infection test and the location information of each client. At this time, the client who provides the information used for the generation of respiratory infection management information includes the client who has applied for the respiratory infection test. That is, the respiratory infection management information may include information of the client who has requested a test but has not yet generated a test result.

The mobile terminal 200 may receive the test result from the central management server 100 or the test agency terminal 300. The test result is provided as negative or positive determinations. If the test result is positive, the central management server 100 may additionally provide information on the treatment agency for treatment of the client and/or transport information for transporting the client to the treatment agency.

According to one embodiment of the present invention, the test result of the respiratory infection may be received within 24 hours from the time the test is requested. According to another embodiment of the present invention, the test result of the respiratory infection may be received from the time the test is requested until the next day. According to another embodiment of the present invention, the test result of the respiratory infection may be received within the same day from the time the test is requested. According to another embodiment of the present invention, the test results of the respiratory infections may be received within 2 days from the time the test is requested.

Even when the client receives the test result of the negative determination or the cure determination of the treatment of the infection, the client continuously transmits the health status information (for example, the questionnaire) and the location information through the application of the mobile terminal 200 to the central management server 100. The health status information may be provided on a daily basis, on a weekly basis, etc. according to various embodiments. The location information is continuously/periodically provided to the central management server 100 in real time even during the same period.

The period in which the health status information is provided may be extended to 3 weeks in units of 1 week, according to an exemplary embodiment. According to another embodiment, the risk of the respiratory infection may be extended on a weekly basis until the end of the risk. According to another embodiment, the central management server 100 may provide the health status information for a period set as needed.

After the positive test result, even when the treatment at the treatment agency is completed and cured, the client continuously transmits the health status information (eg, questionnaire) and the location information through the application of the mobile terminal 200 to the central management server 100. The health status information may be provided on a daily basis, on a weekly basis, etc. according to various embodiments. The location information is continuously/periodically provided to the central management server 100 in real time even during the same period.

According to one embodiment of the present invention, when the client's respiratory infection is maintained as negative for a period of 3 weeks after the test result is determined as negative or the respiratory infection is determined as cured, the provision of location information from the client's mobile terminal may be canceled.

According to another embodiment of the present invention, when the client's respiratory infection is maintained as negative until a period of two weeks after the test result is determined as negative or the respiratory infection is determined as cured, the provision of location information from the client's mobile terminal may be canceled.

According to another embodiment of the present invention, when the client's respiratory infection is maintained as negative until a period of 4 weeks after the test result is determined as negative or the respiratory infection is determined as cured, the provision of location information from the client's mobile terminal may be canceled.

The central management server 100 may be a central control means capable of collecting all information in the mobile terminal management system. The central management server 100 is connected to the mobile terminal 200, the test agency terminal 300, the treatment agency terminal 400, and the like through the communication network. The central management server 100 stores information provided by each of the terminals 200, 300, and 400. The central management server 100 may provide information provided by each terminal 200, 300 and 400 to other terminals. In this case, the information provided to the other terminal may be the same as the received information or may be processed information.

The central management server 100 may select or cancel a test agency and a treatment agency according to a rule or matter determined through a national agency. The central management server 100 may provide information to a newly selected test agency, and may stop or limit information provision and/or information reception to a terminal of the test agency to be deselected.

The central management server 100 may provide information to a newly selected treatment agency terminal, and may stop or limit information provision and/or information reception to a terminal of the treatment agency to be deselected.

The central management server 100 may receive the request for the respiratory infection test and the location information received from the client's mobile terminal 200. The central management server 100 may request the respiratory infection test of the client by selecting any one of the test agencies when there are a plurality of test agencies.

The central management server 100 requests the selected test agency terminal 300 to test the client according to the client's request for the respiratory infection test. The central management server 100 may provide the client's information upon the client's request for the test, and the provided information may include personal information such as name, social security number, address, phone number, age, and gender. In addition, the provided information includes the delivery information for the client to receive the sampling kit.

The central management server 100 may continuously receive information on delivery and collection of the sampling kit from the test agency 300 that delivers the sampling kit to the client. The central management server 100 provides information on delivery and collection of the received sampling kit to the mobile terminal 200 of the client.

The central management server 100 receives the test result performed by using the specimen of the sampling kit recovered from the test agency terminal 300. The test result may include positive or negative determination of the client's specimen.

The central management server 100 continuously receives the location information from the application of the mobile terminal 200 even when the client's test result is voice and generates movement information. The movement information generated using the location information is used to generate the respiratory infection management information. In addition, the central management server 100 may receive a questionnaire about the respiratory infection input to the application of the mobile terminal 200 and check whether the client is infected.

Even if it is negative after the respiratory infection test, the client's health status information is provided through the questionnaire for a certain period of time, and the client's location information is also continuously provided during this period.

According to an embodiment, the certain period during which the central management server 100 is provided with information on the health status of a client may be extended to 3 weeks in units of 1 week. According to another embodiment, the risk of the respiratory infection may be extended on a weekly basis until the end of the risk. According to another implementation example, the central management server 100 may designate and use the certain period in various forms as needed.

The central management server 100 provides the client's test result to the mobile terminal 200. The central management server 100 may provide the client's information to the treatment agency terminal 400 when the client's test result is positive. The provided information may include, for example, personal information such as name, social security number, address, phone number, age and gender. In addition, the provided information includes address information for transport of a positive-determined client to a treatment agency.

The central management server 100 receives transport information of a patient (positive determination client) provided from the treatment agency terminal 400 and provides it to the client's mobile terminal 200. The client may proceed with preparations for hospitalization in the treatment agency according to the transport information provided. That is, the treatment agency terminal 400 may provide information generated in the process of transporting the patient to the treatment agency, the process of initiating the treatment of the patient, and the process of generating the treatment result of the patient to the central management server 100.

The central management server 100 receives treatment information of the patient (positive determination client) provided from the treatment agency terminal 400. The treatment information includes information on a prescription drug used for the treatment of the patient, information on the administration of the prescription drug, information on treatment using the prescription drug, and prognostic information according to the administration and/or treatment of the prescription drug.

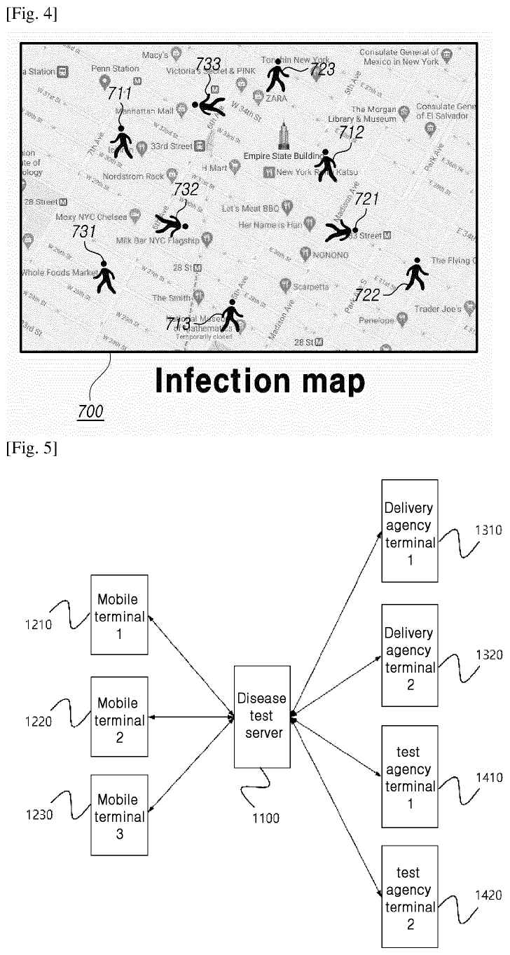

The central management server 100 may generate the respiratory infection management information using the test result and the location information of the client. The respiratory infection management information is information generated by the national agency in order to minimize the spread of the respiratory infections and to perform rapid quarantine. According to one embodiment, the respiratory infection management information is, for example, an infection map. In the infection map, the movement information generated by accumulating the location information of the client may be displayed on the digital map. The movement information displayed on the digital map may include time information according to the movement of the location according to the location information. The infection map may refer to the description of FIG. 4.

The test agency terminal 300 is a terminal that is each held by a single test agency or a plurality of test agencies, and is capable of providing and receiving information to the central management server 100.

The test agency terminal 300 is a terminal of a test agency among a plurality of test agencies selected by the central management server 100. When the test agency terminal 300 is requested to be tested for the respiratory infection, the test agency may perform the respiratory infection test through the process of sampling the client's specimen into the sampling kit, the process of initiating the respiratory infection test on the sampling kit that has been sampled, and the process of generating test results of the respiratory infection.

The test agency terminal 300 may provide the sampling kit for the respiratory infection test to the client by using the client's information provided from the central management server 100. In one example of the provided methods, the client delivers the sampling to the desired location. The client's desired location may vary, for example, client's home, company, or current residence. The central management server 100 may request delivery of the sampling kit by selecting the test agency close to the location where the delivery of the sampling kit designated by the client is desired.

In addition, in another embodiment of the method of providing the sampling kit, there is a method of providing the sampling kit to a place other than the test agency and collecting the specimen from the client. For example, the sampling kit may be provided at easily accessible locations such as airports, passenger terminals, and train stations. The provision of the sampling kit at this time may be provided prior to the client's request for the respiratory infection test, and if the client's request for the respiratory infection test occurs, it may be provided through delivery.

The test agency may require the specimen from the client to be collected by participating personnel for delivery or retrieval of the sampling kit. According to one embodiment of the present invention, when the sampling kit is delivered from the test agency to a personal place such as client's home, company, or current residence, the client is set to perform self-sampling. However, if necessary, a specimen collector who can assist with specimen collection for the client who have difficulty in the self-sampling is required to participate in the delivery or collection of the sampling kit, so that the sampling is collected by the client at the time of delivery or collection of the sampling kit.

According to another embodiment of the present invention, the test agency or the national agency may have a sampling facility in a designated major facility to perform the sampling through a visit of a client. The main facilities include airports, passenger terminals, train stations, and other facilities for foreign and foreign residents to enter the country. In addition, the major facilities may include stadiums, government-affiliated facilities, and the like.

The clients may visit the major facilities and sample the specimen. According to one embodiment, the self-sampling may be possible in the major facilities. According to another embodiment, the specimen collector resides in the major facility to collect the specimen from a visiting client. It may be also possible to collect the specimen through the self-sampling and sampling through the specimen collector in the major facilities.

The test agency terminal 300 provides delivery information and collection information of the sampling kit provided to the client to the central management server 100. The central management server 100 provides the delivery information or the collection information to the mobile terminal 200 of the client.

The test agency terminal 300 performs the respiratory infection test using the sampling kit accommodating the collected specimen of the client. The respiratory infection test is performed using one or more test equipment provided in the test agency, and the test result generated according to the test performed by the test equipment are provided to the test agency terminal 300. The test agency terminal 300 provides the test result to the central management server 100.

The treatment agencies have facilities to treat patients with the respiratory infection, and may include a hospital and a public health center designated by the national agency. The treatment agency terminal 400 is a terminal capable of providing or receiving information from the treatment agency to the central management server 100.

The treatment agency terminal 400 may be held by a single treatment agency or a plurality of treatment agencies, respectively, and is a terminal 400 of a treatment agency among a plurality of treatment agencies selected by the central management server 100. The treatment agency terminal 400 may transfer and treat the client to the treatment agency using information of the client whose test result of the respiratory infection is determined as positive using the client's information provided from the central management server 100.

When the treatment agency terminal 400 provides transport information for transporting the client to the hospital to the central management server 100, the central management server 100 provides the transport information to the mobile terminal 200 of the client.

The means for transporting the client to the treatment agency includes various means of transportation. For example, it is possible to use an ambulance included in the treatment agency or the national agency. If necessary, the client may be transported to the treatment agency using a vehicle designated by the national agency.

The treatment agency terminal 400 may provide information generated in a process for treatment of the client admitted to the treatment agency to the central management server 100. The process for treatment includes a process of transporting the patient to the treatment agency, a process of initiating the treatment of the patient, and a process of generating a treatment result of the patient. The treatment information generated in the process of treatment includes information on prescription drugs used for the treatment of patients, information on prescription drugs, treatment information using prescription drugs, and prognosis information according to the administration and/or treatment of prescription drugs.

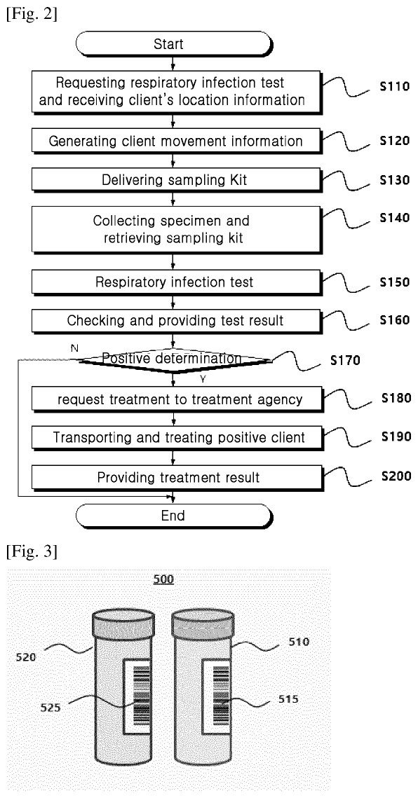

FIG. 2 is a flowchart for illustrating a method of managing a respiratory infection performed using the mobile terminal management system according to the first aspect of the present invention.

As shown in FIG. 2, the central management server included in the mobile terminal management system requests the respiratory infection test and receives the client's location information from the client's mobile terminal at S110.

The client may be a suspected patient with the respiratory infection, or may be a general public who wants to actively check for the respiratory infection. An application (e.g., App.) capable of providing information to the central management server by accessing the mobile terminal management system is installed on the mobile terminal owned by the client.

If the application is not installed on the mobile terminal, the national agency or an agency operating the mobile management server may install the application on the mobile terminal using an application installation file provided through various paths.

The application of the mobile terminal connected to the central management server through the communication network provides location information that may be generated in the mobile terminal to the central management server. The location information may be generated as respiratory infection management information together with the test result of the client in the central management server. The respiratory infection management information may be, for example, an infection map.

Therefore, it is possible for the application of the mobile terminal to provide the client's location information to the central management server in real time, continuously and/or periodically. The client provides the central management server whether or not to consent to the provision of location information, and if agreed, the location information may be provided.

The central management server receives the request for the client's respiratory infection test and the location information, and generates movement information of the client using the received location information at S120.

The movement information generated by the central management server may be used to generate respiratory infection management information. One embodiment of the respiratory infection management information is as shown in FIG. 4.

As shown in FIG. 4, a map may be displayed on the mobile terminal centering on the current location of the client. Locations of other clients indicating various conditions may be displayed on the map. According to an embodiment, the locations of clients 721, 722, and 723 currently undergoing the respiratory infection test may be displayed. According to another embodiment, the positions of the clients 711, 712, and 713 determined as positive after performing a current respiratory infection test may be displayed. Most of the clients that have been determined as positive may be located in a quarantine facility, but clients whose self-isolation and treatment are proceeding after the positive determination may be displayed at the current location. According to another embodiment, there may be displayed the location of the clients 731, 732, and 733 whose determination is negative after the respiratory infection test is performed or and treatment has been completed after the respiratory infection test is performed. Even for clients whose determination is negative and/or treatment have completed, additional observations are required, so the location may be displayed according to the location information collected through the mobile terminal for a period set by the national agency.

In FIG. 4, the current locations of management target clients according to the above embodiment are displayed, but the moving paths of management target clients according to each embodiment may be displayed or displayed as an area of a predetermined width from the management target client, if necessary.

The test agency (test agency terminal) delivers the sampling kit to the client according to the request for sending the sampling kit from the central management server at S130.

The sampling kit is an equipment for sampling the specimen from the client and providing the specimen to the test agency. The test agency delivers the sampling kit to a location desired by the client, and collects the sampling kit that has been sampled to the test agency.

Information on delivery and collection of the sampling kit may be generated by the test agency terminal, provided to the central management server, and then provided to the client's mobile terminal.

After receiving the sampling kit and performing sampling of the specimen, the client requests collection (retrieval) of the sampling kit at S140.

In the case of the self-sampling, the sampling method of the specimen is a method of collecting the specimen by the client. If the sampling of the specimen is a general sampling kit, it is a method that the client's specimen may be collected by a third party. At this time, the third party may be a specimen collector who participated in the delivery or a specimen collector who visits to collect the sampling kit. If necessary, the specimen may be collected through the specimen collector located in a separate location. At this time, the client must move to the separate location, and the separate location may be a location where a medical worker capable of collecting specimens is located.

According to one embodiment of the present invention, in the case of the self-sampling, it is a method of collecting the specimen through oral sampling of the client, and in the case of the general sampling, it may be a method of collecting the specimen through a nasopharyngeal swab of the client.

The test agency performs the respiratory infection test using the retrieved specimen at S150.

The respiratory infection test is performed using the test equipment provided in the test agency, and the test equipment may include a PCR (polymerase chain reaction) device.

The test agency provides the test result to the central management server, and the central management server checks the test result and provides the test result to the client at S160.

The central management server determines the test result provided by the test agency at S170. The test result may include a positive determination or a negative determination. The central management server performs the following steps differently depending on whether it is negative or positive.

If the test result is determined as positive, the central management server may request the client's treatment to the treatment agency while performing from step of S180.

Additionally, the central management server may generate predicted contact information using information of another user determined to have contacted the client by using the client's location information received from the mobile terminal.

When the client's test result of the respiratory infection is positive at S170, the central management server that stores the location information continuously provided from the client's mobile terminal generates predicted contact information corresponding to the stored client's location information.

As one example, the predicted contact information may be generated using information of another connected mobile terminal using terminal access information of the mobile communication base station where the client's mobile terminal is located.

For example, while a client carrying a mobile terminal moves, the mobile terminal continuously maintains a connection state with a neighboring mobile communication base station, and when the mobile terminal moves away from any one mobile communication base station, a new connection state is maintained with other mobile communication base stations instead of the mobile communication base station with which the existing connection state is maintained. Through this, the mobile communication base stations may generate the location information of the mobile terminal through the connection status information with the mobile terminal, and the central management server generate the predicted contact information using the information of the other mobile terminal connected at the same time together with the mobile terminal. In this case, the connection status information may be provided from the mobile communication base station to the central management server, or may be provided from the mobile terminal connected to the mobile communication base station to the central management server.

As another embodiment, the predicted contact information may be generated using information connected to another terminal through a communication method such as Bluetooth and Near Field Communication (NFC) provided by the tester's mobile terminal.

For example, a mobile terminal may transmit/receive signals to and from other mobile terminals through a Bluetooth communication method. In this case, information of other terminals located in the vicinity of the mobile terminal may be received by the mobile terminal, and an application installed in the mobile terminal may provide the received information of the other terminal to the central management server to generate the predicted contact information.

In addition, when the mobile terminal communicates with the terminal using short-range wireless communication, the mobile terminal may receive the terminal information, and the application installed in the mobile terminal provides the received terminal information to the central management server. The central management server identifies a terminal according to the received terminal information, and obtains information of another mobile terminal recognized by the terminal, thereby generating the predicted contact information using the obtained information of another mobile terminal.

The central management server may provide a signal for requesting the respiratory infection test to the mobile terminal of the predicted contact, as contact with the respiratory infection is expected, using one or more predicted contact information generated as in each embodiment.

The central management server may display one or more expected contact information generated as in each embodiment on an infection map, which is the respiratory infection management information.

The central management server may reflect the generated expected contact information in the calculation of the infection reproduction number (R). The infection reproduction number (R) is defined as the average number of people in a population with an infectious patient who transmits the infection during the possible transmission period. R means a value greater than or equal to 0, and if R>1, it means that at least one person may be infected additionally, meaning that the infectious disease spreads within the population and the epidemic continues. In addition, if R<1, it means that the prevalence is reduced and the incidence of infectious diseases is reduced. When R=1, it means continuous occurrence, and endemic disease corresponds to the case.

The infection reproduction number (R) may be expressed as R=p×c×d, and p is the probability of infection, which can be reduced through the use of therapeutic agents, masks and the like. c is the contact rate, which can be reduced by the effect of strengthening social distancing and the like, and d is the duration for spreading the infection, which can be reduced through rapid isolation of the patient by increasing the diagnostic test volume. Accordingly, the government is trying to prevent the spread of infectious diseases through quarantine measures such as the use of masks, the social distancing, and the rapid diagnostic tests. Therefore, the calculation of R becomes an important index for evaluating the effectiveness of quarantine policies and monitoring the spread of infection.

The infection reproduction number may be classified into basic infection reproduction number (R0), effective infection reproduction number (Re), and time-varying infection reproduction number (Rt), and each feature of them is as follows.

The basic infection reproduction number (R0) refers to the infection reproduction number when an infection disease occurs in a population that is not immune to a certain infection disease (new infection disease, unvaccinated) and management and intervention for this infection disease have not yet been introduced. As an index indicating biological infectivity, it is mainly calculated at the early epidemic stage of the infection diseases, and R0 is not a unique value given for each infection disease, but is known to show similar values for the same infection disease.

When the outbreak continues past the initial pattern of disease, the effective infection reproduction number (Re) is refers to the infection reproduction number that takes into account personal hygiene management (reinforcement of hygiene, social distancing, etc.) and social measures(quarantine, containment, school restrictions, large-scale gatherings, etc.) when the outbreak continues after the initial pattern of disease. In fact, the “infection reproduction number” after the early infection disease epidemic refers to the effective reproduction number, and is used to track changes in infection power over time or to determine the short-term effects of interventions.

Among the effective infection reproduction number (Re), the average infectivity of the population at a specific time point, which can be repeatedly calculated over time, is defined as the time-varying reproduction number (Rt), and at time t, Rt can be calculated in two ways of instantaneous reproduction number and case reproduction number. The instantaneous reproduction number measures the propagation at a specific time point, while the case reproduction number measures the propagation by individuals in a specific cohort group. The case reproduction number is useful for retrospective analysis of how infected individuals at different times contributed to the spread (Myungsoo Yoo, Yeonju Kim, Sujin Baek, Donghyuk Kwon. “Concept of infection reproduction number and changes according to quarantine policy”, Weekly Health and Disease/Vol. 14, No. 6 (Feb. 4, 2021)).

When the test result is determined as positive, the central management server requests the treatment agency to treat the respiratory infection of the client at S180.

The treatment agency receives the client information included in the request for treatment of the respiratory infection.

The treatment agency may transport the client to the treatment agency and initiate his/her treatment using the received client's information at S190.

The client may be transported through a transport means provided in the treatment agency, but if necessary, the transport may be transported using the transport means included in the national agency or the transfer means designated by the national agency.

The treatment agency initiates treatment of the respiratory infection to the transported client, but provides client's transport information and treatment information to the central management server. The treatment information may include information on medication administration for the client's treatment, information on medication treatment, and prognosis information after medication. If symptoms of the respiratory infection disappear from the client, additional respiratory infection tests are performed to determine whether or not to cure.

Thereafter, the treatment agency provides the client's treatment result to the central management server at S200.

The central management server may modify the client's infection management according to the treatment result. Even if the test result is determined as cured, it continuously receives the client's health status information and the location information for the period specified by the national agency, and performs continuous infection management for the client.

Even if the test result is determined as negative, the central management server continuously receives the client's health status information and the location information for a period specified by the national agency, and performs continuous infection management for the client.

Even when the test result is determined as negative or cured, the client continuously generates the health status information and the location information through the application of the mobile terminal according to the infection control policy of the national agency and provides it to the central management server.

FIG. 3 is an exemplary view illustrating a sampling kit according to an embodiment of the present invention.

As shown in FIG. 3, the sampling kit 500 is a kit for oral sampling, and one container 510 is a specimen transport container 510 for receiving a specimen through the oral sampling, and the other container 520 is a gargle container 520 containing a solution for the oral sampling (e.g., physiological saline, etc.).

Each container is included in the delivered sampling kit and provided to the client, and the client may complete the sampling by putting the specimen through self-sampling (gargle) in the specimen transport container 510.

The specimen transport container 510 may include a transport medium for preventing the specimen from being deteriorated or inactivating the specimen during the collection period to the test agency.

In addition, although not shown in the drawing, the delivered sampling kit may further include a collection equipment for collecting the specimen transport container 510. Since the specimen is a pathogen that causes the respiratory infection, there is a risk even if the pathogen is inactivated through the transport medium. Accordingly, the container 510 for transporting the specimen is packaged through the collection equipment that is necessarily tightly sealed, and the collection equipment in which the container 510 for transporting the specimen is accommodated is provided to the test agency.

Meanwhile, each of the specimen transport container 510 and the gargle container 520 includes a barcode for identification of the client and identification of the provided sampling kit. The barcode may be included prior to shipment of the sampling kit by the test agency.

The barcode may be implemented differently according to various implementations. For example, in the case of a means including identifiable information such as a QR code, an NFC chip, a figure, a character, or a picture, it may be replaced with any one.

In addition, the packaging container for delivery, the collection equipment, etc. may also include a barcode or the like that may include the information of the client.

FIG. 3 shows the configuration of the sampling kit for the oral sampling among the self-sampling kits according to an embodiment of the present invention, and in other various embodiments, the configuration of a container included in the sampling kit may be one, three or more than four.

In addition, the sampling method can be divided into a self-sampling method and a sampling method by a specimen collector to construct the sampling kit.

In another embodiment of the present invention, the sampling kit includes a container for transporting the specimen. The container for transporting the specimen may be provided as an empty container or may be provided in a state containing a transport medium for the specimen. The transport medium for the specimen may be provided in a separate container.

In the present specification, the term ‘the container for transporting the specimen includes transport medium for the specimen’ may refer not only to the case where the transport medium for the specimen is contained in the container for transporting the specimen, but also to the case where the transport medium for the specimen is contained in a separate container.

The transport medium for the specimen is used for the purpose of stabilizing the test object in the specimen, inactivating microorganisms in the specimen, lysis of the specimen, preventing deformation of the specimen, or preventing spoilage of the specimen. The transport medium for the specimen may be liquid or solid.

If the specimen is sputum, saliva, urine or feces, the container for transporting the specimen may be provided as an empty container. In the case of the above specimen, the transport medium for the specimen may be used, if necessary.

The sampling kit may additionally include a tool for collecting the specimen. The tool for collecting the specimen include, for example, a swap tool, a syringe, an eye dropper, a container for temporarily storing the specimen during the sampling process, or a container containing a composition for collecting the specimen and the like. The sampling kit may include a composition for collecting the specimen. For example, the composition for collecting the specimen is an oral and/or throat wash-out solution. The composition for collecting the specimen may be provided in a container.

The sampling kit may additionally include a sealed container capable of sealing the container for transporting the specimen.

The sampling kit may be configured to collect the specimen such as human oral and/or throat wash-out products, saliva, sputum, oral swabs, nasal swabs, blood, urine, feces, stool and the like.

The sampling kit may include a specimen collection composition and/or a specimen delivery medium.

According to one embodiment, the sampling kit includes a specimen transport container including a swap tool and a transport medium for the specimen.

According to one embodiment, the sampling kit includes a container for transporting the specimen comprising a dropper and a transport medium for the specimen.

According to one embodiment, the sampling kit includes a container containing an oral and/or throat wash-out solution. The container containing the oral and/or throat wash-out liquid may be reused as a container for transporting the specimen.

According to one embodiment, the sampling kit includes a container for transporting the specimen comprising an oral and/or throat wash-out solution and the transport medium for the specimen.

Although the above embodiment of the present invention has described an embodiment of delivering the sampling kit to a place requested by the client, the present invention may be applied to other embodiments as follows.

An application (App.) that may use the mobile terminal management system may be installed on the mobile terminals of foreign entrants entering Korea. The application provides location information of the foreign entrants to the central management server, and the central management server may perform infection management for each of the foreign entrants.

The foreign entrants may request the respiratory infection test (eg, COVID19 or SARS CoV-2) through the installed application. The respiratory infection test may be paid as needed, and payment may be performed through the application installed on the mobile terminal.

When a request of the respiratory infection test is completed through the application, a specimen may be sampled at a site located in an airport or a passenger terminal where the entrance procedure is in progress. The sampling in the field may be the self-sampling if necessary, and in general, the specimen may be collected from the medical worker who may collect the specimen.

Even in the case of performing the respiratory infection test through sampling at the site, in one embodiment of the present invention, the test result may be provided to the foreign entrants within 24 hours. In another embodiment of the present invention, the test result may be provided to the foreign entrants within the same day. In another embodiment of the present invention, the test result may be provided to the foreign entrants the next day. In another embodiment of the present invention, the test result may be provided to the foreign entrants within 2 days.

If the test result of the foreign entrant is determined as positive, the central management server requests the treatment of the foreign entrant to the treatment agency, and the treatment agency quarantines and treats the foreign entrant.

Even if the test result of the foreign entrant is determined as negative, the health status information (eg, questionnaire) and the location information are continuously provided to the central management server 100 through the application of the mobile terminal 200 of the foreign entrant. The provided health status information may be provided on a daily basis, on a weekly basis, etc. according to various embodiments. The location information is also provided to the central management server 100 continuously/periodically in real time during the same period.

The health status information and the location information are continuously provided to the central management server for a period specified by the national agency, and the central management server continuously receives the client's health status information and location information, and performs continuous infection management for the client.

At this time, the period designated by the national agency is up to 3 weeks in units of one week according to an embodiment, and the health status information and the location information are provided to the central management server. According to another embodiment, it is up to two weeks in units of one week, and the health status information and the location information are provided to the central management server. According to another embodiment, it is up to 4 weeks in units of one week, and the health status information and the location information are provided to the central management server.

According to an embodiment of the present invention, if the test result of the foreign entrant is determined as negative or his/her respiratory infection is maintained as negative for a period of 3 weeks after the test result is determined as cured, the provision of the location information from the client's mobile terminal may be canceled.

According to another embodiment of the present invention, if the test result of the foreign entrant is determined as negative or his/her respiratory infection is maintained as negative for a period of 2 weeks after the test result is determined as cured, the provision of the location information from the client's mobile terminal may be canceled.

According to another embodiment of the present invention, if the test result of the foreign entrant is determined as negative or his/her respiratory infection is maintained as negative for a period of 4 weeks after the test result is determined as cured, the provision of the location information from the client's mobile terminal may be canceled.

The mobile terminal management system for the respiratory infection management according to the first aspect of the present invention may be made by the following embodiment.

According to the first aspect of the present invention, a central management server for managing respiratory infections may use a memory, a processor, and one or more programs stored in the memory and be configured to be executed by the processor. When the one or more programs are executed by one or more processors, it comprises instructions to perform: receiving, by the central management server, a request for a respiratory infection test from the client's mobile terminal; receiving, by the central management server, location information from the mobile terminal to generate movement information; requesting, by the central management server, the respiratory infection test for the client to a terminal of a test agency; receiving, by the central management server, a test result of the respiratory infection of the client from the terminal of the test agency; and generating, by the central management server, the respiratory infection management information using the movement information and the test result.

The mobile terminal management system for the respiratory infection management according to the first aspect of the present invention may be made by the following other embodiments.

According to the first aspect of the present invention, a non-transitory computer-readable storage medium stores an instruction which performs a method for managing respiratory infections by one or more processors in a computing device when executed by the one or more processors. When the instruction is executed by the one or more processors, the computing device performs: receiving, by the central management server, a request for a respiratory infection test from the client's mobile terminal; receiving, by the central management server, location information from the mobile terminal to generate movement information; requesting, by the central management server, the respiratory infection test for the client to a terminal of a test agency; receiving, by the central management server, a test result of the respiratory infection of the client from the terminal of the test agency; and generating, by the central management server, the respiratory infection management information using the movement information and the test result.

The mobile terminal management system for the respiratory infection management according to the first aspect of the present invention may be made by the following embodiment.

According to the first aspect of the present invention, a terminal is connected to the above-described central management server of through a communication network and installed with an application that performs the function of a mobile terminal in each step of performing a method of managing respiratory infections.

FIG. 5 is a view schematically illustrating a configuration of a disease test system through remote specimen sampling according to a second aspect of the present invention.

As shown in FIG. 5, mobile terminals 1 to 3 are terminals 1210, 1220, and 1230 carried by a plurality of users. The mobile terminal may be a personal communication device such as a smartphone on which an application capable of accessing the disease test server 1100 is installed.

The disease test server 1100 may be accessed through a network, through the Internet, by one or more mobile terminals 1210, 1220, and 1230. The disease test server 1100 (hereinafter referred to as ‘server’) receives information from the application installed in one or more mobile terminals 1210, 1220, and 1230, and provides information generated by the server 1100 or external information to each application of the selected mobile terminal among the mobile terminals 1210, 1220 and 1230.

Hereinafter, information, signals, data, etc. transmitted between the mobile terminal and the server 1100, or between the delivery agency terminals 1300, 1310 and 1320, and the test agency terminals 1400, 1410 and 1420 are transmitted and received through the dedicated application installed on the mobile terminal. Therefore, the server 1100, the delivery agency terminals 1300, 1310 and 1320, and the test agency terminals 1400, 1410 and 1420 may be configured to transmit or receive information, signals, data, etc. to or from the dedicated application installed in the mobile terminal.

In addition, if necessary, the delivery agency terminals 1300, 1310 and 1320 and the test agency terminals 1400, 1410 and 1420 are not allowed to directly communicate with the mobile terminal, and may receive information about a mobile terminal or a user by connecting with the server 1100, as shown in FIG. 5.

The mobile terminals 1200, 1210, 1220, and 1230 may receive GPS signals and generate GPS-based location information. Accordingly, when the application installed in the mobile terminal transmits the user's location information to the server 1100, it may provide the GPS-based location information according to the GPS signal.

If necessary, the location information provided by the mobile terminals 1200, 1210, 1220, and 1230 may transmit location information indicating an administrative area such as an address.

The delivery agency terminals 1300, 1310, and 1320 are terminals included in the delivery agency, and may select and deliver a sampling kit to a user according to sampling kit information included in the instruction information transmitted from the server 1100. The location information of the user for delivery is location information such as the GPS-based location information mentioned above or an address that may indicate an administrative area.

The test agency terminals 1400, 1410 and 1420 collect the sampling kit containing the user's specimen, obtain the test result analyzing the specimen with one or more test equipment, and transmit it to the server 1100.

The test agency terminals 1410 and 1420 are terminals respectively included in one or more test agencies, and according to the user's request for disease test, the test agency terminals of at least one or more selected test agencies in the server 1100 may collect the sampling kit and perform the test.

In the present application, the delivery agency and the test agency are described separately as separate agencies, but the delivery agency and the test agency may be the same agency, and the name at this time may be combined and used as a test agency. That is, the delivery of the sampling kit, collection of the sampling kit, and test of the specimens contained in the sampling kit may be performed by the test agency. In this case, the delivery agency terminal may be replaced with the test agency terminal to perform both functions.

The server 1100 may transmit the received test result to the mobile terminal when the test result is received from the terminal of the selected test agency among the test agency terminals 1410 and 1420.

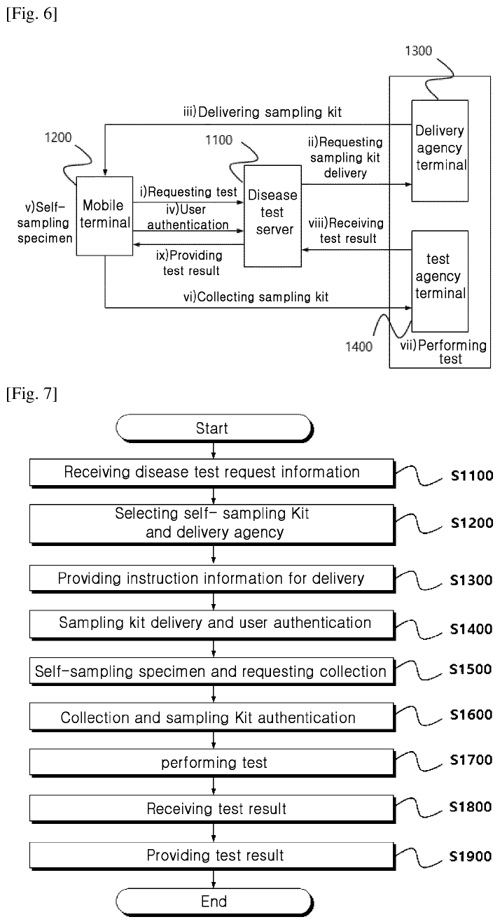

FIG. 6 is a view schematically illustrating a configuration of a disease test system through the remote specimen sampling according to an embodiment of the second aspect of the present invention. FIG. 7 is a flowchart illustrating a method for testing a disease through the remote specimen sampling according to the second aspect of the present invention.

As shown in FIGS. 6 and 7, a user who shows symptoms of a disease transmits disease test requests information to the server 1100 by using an application installed in the mobile terminal 1200 at S1100.

The disease test request information includes personal information such as the user's name, social security number, phone number, and location information such as address. The mobile terminal 1200 may encrypt the personal information through a security module that may be used in the application and transmit it to the server 1100.

In particular, since the user is located remotely from a medical agency or a screening test center where the specimen may be collected, the location information such as address information must be input more accurately.

The disease test request information may include one or more information input by the user such as symptoms of the disease, body temperature, a predicted infection route, and a movement route that the user has moved according to the predicted infection route. In addition, the disease test request information may include information for directly selecting the sampling kit for testing symptoms of the disease from the user. The user may input detailed information required by the application for proper infection prevention and treatment and provide it to the server 1100.

The server 1100 receives the disease test request information from the mobile terminal 1200 and selects the sampling kit capable of testing a disease for a user's symptoms in response to the disease test request information. Since the sampling kit is owned by at least one delivery agency among a plurality of delivery agencies, the server 1100 selects one delivery agency from among delivery agencies holding the sampling kit and provides instruction information for requesting delivery of the sampling kit to a remotely located user at S1300.

That is, the delivery agency selected by the server 1100 has the sampling kit corresponding to the user's disease. The server 1100 may select a delivery agency close to the user's location information.

In one embodiment, the server 1100 may directly select a sampling kit to be selected according to the symptoms of the user included in the disease test request information. In other words, it is possible to automatically identify a disease according to the input symptoms and select a corresponding sampling kit.

In another embodiment, after the doctor confirms the user's symptom among the user's disease test request information received by the examination agency terminal 1400 or the delivery agency terminal 1300, a disease and a sampling kit may be selected. The test agency terminal 1400 may provide information on the selected disease and the corresponding sampling kit to the server 1100, and deliver the sampling kit to the user with the approval of the server 1100.

In another embodiment, the server 1100 may select the sampling kit included in the disease test request information by directly selecting the user.

The instruction information transmitted from the server 1100 to the delivery agency terminal 1300 includes information on a sampling kit of a specimen for disease test of a user located remotely and delivery information, which is location information of the user.

The delivery agency terminal 1300 enables the sampling kit according to the received instruction information to be delivered to the user. Delivery of the sampling kit may be performed by personnel directly from the delivery agency. Alternatively, delivery of the sampling kit may be consigned to a separate delivery company so that it may be delivered to a remote user.

Accordingly, when the sampling kit is delivered to the remotely located user, the user authenticates that the sampling kit has been normally received using the mobile terminal 1200 at S1400.

An identifier is included in the sampling kit, and the user recognizes the identifier included in the sampling kit using the mobile terminal 1200 so that the mobile terminal 1200 may generate the identifier information.

The identifier may provide information through a bar code, a QR code, a near field communication chip (NFC chip), or the like. In addition, the identifier includes separate figures, characters, images, electronic data, and the like.

The server 1100 receives the identifier information transmitted from the mobile terminal 1200, compares the sampling kit corresponding to the identifier information with the user, and checks whether the sampling kit is mis-delivered. If the user and the delivered sampling kit do not match, the sampling kit is collected, and re-delivery of the sampling kit is performed if necessary.

In addition, reception of the sampling kit using the identifier may be authenticated at S1400, and user authentication may be additionally performed or only user authentication may be performed if necessary.

The user authentication provides information that may authenticate a legitimate user using the mobile terminal 1200 to a server. The user authentication mechanism used at this time may be performed through one or more authentication methods among a public certificate, a password preset through an application, and biometrics. The password includes pattern recognition, and the biometric recognition includes any one or more of fingerprint recognition, iris recognition, face recognition, and the like.

In addition, the mobile terminal 1200 transmits the generated identifier information to the server 1100, and the server 1100 receives the identifier information. The server 1100 may compare the identifier information with the user of the mobile terminal 1200 to check whether the sampling kit to be provided to the user is correct. If the user and the sampling kit correspond according to the identifier information, the server 1100 performs the following steps. If the user and the sampling kit do not correspond according to the identifier information, the server 1100 may transmit a message for stopping sampling to the mobile terminal 1200 and proceed with a procedure for collecting the delivered sampling kit.

When reception authentication or user authentication of the sampling kit is normally completed at S1400, the server 1100 may provide a user manual of the sampling kit to the mobile terminal 1200.

The instruction manual for the sampling kit includes instructions on how the user uses the sampling kit to collect the specimen and how to use the sampling kit. To this end, the instruction manual includes at least one or more of text, images, and videos.

The server 1100 may provide a user manual directly to the mobile terminal 1200 or indirectly by providing a URL (Uniform Resource Locator) through which the mobile terminal 1200 may access online.

The server 1100 may provide the mobile terminal 1200 with a video manual from among the instruction manuals of the sampling kit. The video manual is a video on how to use the sampling kit according to the disease test requested by the user.

For example, the video may show how to collect the specimen to be collected from specimens such as human oral wash-out results, saliva, sputum, oral swabs, nasal swabs, blood, urine, and stool depending on the user's symptoms and how to use the collection tool and the like.

The moving picture manual may be produced as a single moving picture for the entire process of sample collection, or may be produced as a plurality of moving pictures divided for each process of the sample collection.

In one implementation, the server 1100 may provide a user manual for a sampling kit produced as a single video to the mobile terminal 1200.

In another embodiment, the server 1100 may provide a manual for use of a sampling kit produced as a plurality of videos to the mobile terminal 1200. When the user manual consists of a plurality of moving pictures, the server 1100 may selectively provide a sample according to an order of specimen collection from among a plurality of moving pictures divided into stages in which the user takes a specimen. When the user manual consisting of a plurality of moving pictures is selectively provided to the mobile terminal 1200, the server 1100 may provide it in response to a “sample collection checklist for self-sampling” to be described later.

When the server 1100 provides a video manual corresponding to a sampling kit for self-sampling among various specimens to the mobile terminal 1200, the user may check the provided video manual and uses the received sampling kit to collect the user's specimen.

The server 1100 may provide the mobile terminal 1200 with the specimen collection checklist for the self-sampling. The specimen collection checklist may include:

(i) What to check when the user collects the specimen using the sampling kit, and

(ii) What to check whether the user has performed each process of the specimen collection according to appropriate procedures.

In one embodiment, the server 1100 may provide the specimen collection checklist including (i) to the mobile terminal 1200.

In another embodiment, the server 1100 may provide the specimen collection checklist including (ii) to the mobile terminal 1200.

In another embodiment, the server 1100 may provide a specimen collection checklist including i) and ii) to the mobile terminal 1200.

For example, if the sampling kit received by the user collects a saliva specimen, the server 1100 may provide a specimen collection checklist including the follows to confirm the exact specimen collection of the user.

(i) What to check whether the specimen collection time is early in the morning, before meals, or before brushing.

(ii) What to check whether food or water has been consumed 30 minutes prior to the collection of the specimen

(iii) What to check whether saliva is on the outside of the specimen container when the user spits out the saliva in the mouth into the specimen container, and closes the lid.

(iv) What to check whether information on the user and/or the type of specimen is entered in the specimen container.

The specimen collection checklist is provided to the mobile terminal 1200 according to the type of the specimen for the self-sampling, and the mobile terminal 1200 may transmit the checklist input by the user to the server 1100.

If the server 1100 does not receive the information input in the checklist, or if there is no input of the correct information, the specimen may not be collected.

The information on the user and/or the type of specimen, etc., which is written in the specimen container in the above step (iv), may be directly written by the user, but the information may be printed in advance and sent to the specimen container included in the sampling kit.

For example, the information,

(i) may be written by the user,

(ii) may be pre-written pared or printed in advance by the delivery agency and attached to the specimen container, or

(iii) may be pre-written or printed by the delivery agency, be enclosed in the sampling kit, and be attached by the user.

In addition to the user and/or the specimen type, the information may additionally include information that may identify the user and the specimen, such as the user's name, age, and collection date.

On the other hand, in steps S1300 and S1400, when the specimen is collected at the airport as the user enters Korea from an overseas infected area, steps S1100 and S1200 are normally performed. However, since the sampling kit may be provided by the medical agency near the airport or a screening test center temporarily installed at the airport, the user transmits disease test request information to the server 1100 using the mobile terminal 1200, the server 1100 provides instruction information corresponding to the disease test request information to the terminal of the medical agency near the airport or the screening test center installed at the airport, so that the user may visit the relevant medical agency or screening test center to collect the user's specimen.

The user who has finished collecting the specimen may return home or move to the quarantine facility, and the server 1100 may receive a collection request transmitted from a terminal of the medical agency or the screening test center near the airport.

Upon receiving the collection request, the server 1100 performs the same procedure from S1600, but the user may visit the corresponding medical agency or the screening test center to collect the sampling kit containing the user specimen.

The user collects the specimen from his/herself in the provided sampling kit, and requests the server 1100 to collect the sampling kit through the mobile terminal 1200 at S1500.

The specimen collection may be performed by the user's own specimen sampling method, and the user's specimen may be collected by a person who delivers the sampling kit from the delivery agency. In other words, since the sampling kit using the washing-out method of the user's oral cavity allows the user to relatively easily collect the specimen, only the sampling kit is delivered, thereby requesting the self-sampling from the user. However, other sampling methods, such as throat swab and nasal swab, are difficult for the user to self-sample, so the user's specimen is sampled by the person who delivers the sampling kit from the delivery agency.

On the other hand, the specimens collected from the user's self-sampling or manpower sampling includes at least one of the user's oral wash-out product, saliva, sputum, oral swab, nasal swab, blood, blood, urine or stool.

In step S1500, the collection request transmitted to the server 1100 through the mobile terminal 1200 includes location information of the user for collection of the sampling kit. The user's location information may be GPS-based location information obtainable from the mobile terminal 1200 or location information including an address input from the user. Accordingly, the server 1100 selects the test agency capable of collecting the user's sampling kit according to the collection request received from the mobile terminal 1200, and provides an instruction information for collecting the sampling kit to the terminal 1400 of the selected test agency. The instruction information includes information on the sampling kit and the location information of the user.

The test agency terminal 1400 dispatches collection personnel and equipment for collecting the user's sampling kit to the user's location information according to the instruction information received from the server 1100. Since the user collected the specimen due to the expectation of a specific disease, the user's specimen is at risk of spread of pathogens when the user's disease is not determined as positive or negative so that the specimen must be delivered to the test agency by using the collection personnel and equipment.

The test agency terminal 1400 collects the sampling kit accommodating the specimen using the user's location information, recognizes an identifier included in the sampling kit, and generates identifier information. The test agency terminal 1400 transmits collection completion information including the generated identifier information to the server 1100 to receive a confirmation of the sampling kit delivered to the corresponding user.

The server 1100 checks the sampling kit to be collected using the collection completion information including the identifier information transmitted from the test agency terminal 1400 and provides the test of the sampling kit to the test agency terminal 1400.

The test agency terminal 1400 performs the test of the specimen according to a request to proceed with the test of the server 1100 at S1700. The test of the specimen is performed using one or more test devices, and the test result according to the completion of the test of the test device is transmitted to the test agency terminal 1400.

The test device may be a real-time PCR device, and the test result obtained by analyzing the specimen using one or more real-time PCR devices and peripheral devices may be automatically input to the test agency terminal 1400. However, it is not excluded that the test result generated by the test device is input to the test agency terminal 1400 by the tester.

Two types of Internal Control (IC) may be used when testing the user's specimen included in the collected sampling kit. It is used to verify the validity of the specimen collected through the user's self-sampling, the extraction process and the validation of the nucleic acid test, and the validation of the nucleic acid amplification process.

At this time, the Internal Control (IC) such as an endogenous IC and an exogenous IC may verify the validation of the entire test process from the sample collection to nucleic acid extraction and amplification. The purpose of use for each IC is as follows.

TABLE 1

| Purpose of use | Endogenous IC | Exogenous IC |
|---|---|---|
| Validation for test of the collected | ◯ | — |
| sample | | |
| Validation for extraction process | ◯ | ◯ |
| and test of nucleic acid | | |
| Validation for nucleic acid | ◯ | ◯ |
| amplification process | | |

In other words, since the endogenous IC may verify the validation of the self-sampled specimen, the test agency uses the endogenous IC to verify the validation of the specimen collected by the user.

When the determination of nonconformity occurs in the endogenous IC test of the sample, the test agency terminal 1400 provides its result of the specimen to the server 1100. The server 1100 provides its result of the specimen to the mobile terminal 1200.

The server 1100 may provide instruction information for requesting re-delivery of the sampling kit to the delivery agency terminal 1300 as necessary.

In the event of the determination of nonconformity in the validation of the extraction process and the validation of the nucleic acid test and the validation of the nucleic acid amplification process during the endogenous IC or exogenous IC testing, the test agency performs a retest using the relevant specimen.

The test agency terminal 1400 provides the test result of the sample whose validity is verified in the internal control material test to the server 1100.

The server 1100 receives the test result provided from the test agency terminal 1400 at S1800. The test result received by the server 1100 may be a test result generated from the test device of the test agency. Alternatively, the test result received by the server may be processed so that a user may easily check the test result generated by the test device of the test agency.

The server 1100 provides the test result to the mobile terminal 1200 at S1900, and if necessary, may provide the test result to a government agency terminal.

The government agency terminal may be a CDC (Centers for Disease Control) that collects and provides information on diseases. The Centers for Disease Control is called as the “Disease Management Headquarters” in Korea.

If the test result for the user's specimen is determined as positive, the server 1100 provides various information such as the followings to the mobile terminal 1200 so that the spread of infectious diseases of the user may be prevented and treatment of the user may be effectively performed:

(i) treatment agency information corresponding to the user's location information for the user's outpatient treatment;

(ii) treatment of the user's disease and/or information on the behavior of the user prior to treatment; and

(iii) transport progress information to transfer the user to the treatment agency.

In addition, if the test result for the user's specimen is determined as positive, the server 1100 may activate an application installed in the mobile terminal 1200 to perform the follows:

(i) receiving movement path information of a user generated by using the location information of the mobile terminal;

(ii) receiving dosage information for prescription drugs entered into the mobile terminal; and

(iii). receiving prognostic information according to the dosage of prescription drugs entered into the mobile terminal.

Alternatively, the application installed in the mobile terminal 1200 may be activated by a user.

In one embodiment, the user faithfully inputs the information requested by the server to the mobile terminal 1200 to treat and prevent the spread of the disease, and the mobile terminal 1200 transmits the input information to the server 1100 do.

In another embodiment, the server 1100 may receive (ii) dosage information for the prescription drug and (iii) prognostic information for the prescription drug administration through the treatment agency terminal (not shown).

When the user's test result is determined as positive, the server 1100 may provide information transmitted from the mobile terminal 1200 to the government agency. Alternatively, information transmitted from the treatment agency terminal may be provided to the government agency. If the user's disease is an infection disease, the government agency continuously monitors the user's condition to block the spread of the infectious disease, and accumulates the data so that it may be used as data for quarantine measures.

Therefore, the application installed on the mobile terminal 1200 informs the user that such personal medical information may be provided to the government agency, and the user consents to this, so that the server 1100 provides the medical information of the user to the government agency.

When the user's test result is determined as positive, the server 1100 may link with the medical agency for treatment of the user. Accordingly, when the user is a severely ill disease, an infection disease, or a disease with inconvenient mobility, the medical agency may provide a vehicle for transport of the user.

When it is determined that the symptoms according to the user's disease are mild, the server 1100 may transmit a message requesting a visit to the medical agency to the mobile terminal 1200.

The sampling kit provided to the user may contain a specimen capable of testing for hereditary diseases and infectious diseases. The infectious diseases include at least one or more infection pathogens of the respiratory infection pathogens, gastrointestinal infection pathogens, and sexually transmitted infection pathogens.

In particular, the respiratory infection pathogens include at least one selected from coronavirus, influenza virus, RSV (respiratory syncytial virus), adenovirus, enterovirus, PIV (parainfluenza virus), MPV (metapneumovirus), bocavirus, rhinovirus (rhinovirus) and Mycoplasma pneumoniae.

For example, the coronavirus is SARS-CoV-2 (severe acute respiratory syndrome coronavirus 2).

In particular, the respiratory infection pathogens that may be tested through the provided sampling kit include COVID19 and RV-EA, and may additionally include RP if necessary. That is, the user may test two pathogens of COVID19 and RV-EA through one sampling kit, and additionally test RP pathogens at the request of the user.

In addition, the gastrointestinal infection pathogens include at least one pathogen selected from adenovirus, rotavirus, norovirus, Shigella, Helicobacter pylori, Campylobacter, Salmonella, and astrovirus.

In addition, the sexually transmitted pathogens include at least one pathogen selected from N. gonorrhoeae, C. trachomatis, U. urealyticum, M. genitalium, M. hominis, T. vaginalis, Gardnerella vaginalis (G. vaginalis), Candida albicans (C. albicans), Herpes simplex virus-type 1 (HSV-1), Herpes simplex virus-type 2 (HSV-2). The Infection pathogens may be viruses, bacteria or fungi.

On the other hand, the server 1100 may provide progress information for each step to the mobile terminal 1200 from the time the disease test request information is received from the mobile terminal until the test result is provided in order to test the user's disease. The information provided in each step includes information on the disease test, information on the current location of the sampling kit to be delivered, information on the delivery agency, information on the test agency, information on user instructions corresponding to the test result, and the like.

The sampling kit includes a container for transporting the specimen. The container for transporting the specimen contains transport medium for the specimen. The sampling kit may additionally include a sealed container capable of sealing the container for transporting the specimen. The sampling kits may be configured to collect the specimen such as human oral wash-out products, saliva, sputum, oral swab, nasal swab, blood, urine, stool, etc.

In particular, the sampling kit capable of collecting the specimen of oral wash-out product include (a) a first container including an aqueous solution and a washing-out solution for wash-out the respiratory pathogens in the oral cavity and/or throat; and (b) a second container including a transport medium containing an inactivating agent for inactivating the respiratory infection pathogen. The aqueous solution of the wash-out solution consists of water or a saline-based solution. The wash-out solution may additionally include at least one component selected from the group consisting of (i) an alcohol selected from the group consisting of ethanol and isopropanol, and (ii) a detergent.

The transport medium including the inactivating agent may include (i) a chaotropic agent. In particular, the transport medium may additionally include at least one selected from the group consisting of (ii) a detergent; (iii) a reducing agent; and (iv) a chelator.

The transport medium may have a function of inactivation by lysis of the respiratory infection pathogens and a stabilizing function of nucleic acid material released from the crushed pathogen.

The oral sampling kit may be sampled by applying the wash-out solution of the first container to the oral cavity and/or throat of a human to gargle, and injecting the resulting gargling solution into the transport medium of the second container.

In addition, the wash-out solution of the first container is applied to the oral cavity and/or throat of the human, (i) brushing or (ii) gargling and brushing, and brushing resultant, brushing resultant and the gargling resultant liquid or brush may be sampled by pouring it into the transport medium in the second container. The sampling kit may additionally include an oral brush or an oral swab, a nasal swab, and the like.

Another aspect of the present invention of the method for testing the disease through the specimen sampling is as follows.

The user inputs disease test request information on the mobile terminal.

The disease test request information provided from the mobile terminal may include information for specifying the user, such as the user's name, social security number, and address.

The disease test request information provided from the mobile terminal may include the name of a related disease that the user wants to be tested. For example, it may be expressed as a respiratory infection disease, a gastrointestinal infection disease, a sexually transmitted infection disease, a disease caused by a genetic variation, etc.

The disease test request information provided from the mobile terminal may include the type of assay that the user wants to be tested. For example, a plurality of assays may be presented on the mobile terminal, and the user may select one assay, two assays, or three or more assays.

According to one embodiment, one assay is configured to detect the presence of one or more pathogens. For example, one assay may be configured to detect the presence of 1 to 50 pathogens, 1 to 40 pathogens, 1 to 30 pathogens, 1 to 20 pathogens, 1 to 10 pathogens, 1 to 5 pathogens, one to two pathogens or one pathogen. One assay may include screening and detecting a group of pathogens (e.g., similar pathogens) in addition to a specific pathogen.

According to an embodiment, the disease test request information is input by the user by inputting into the mobile terminal or by checking an option presented by the mobile terminal.

The server provides instruction information regarding which specimen sampling kit to be provided to which user to the terminal of the provider of the specimen sampling kit using the disease test request information.

According to one embodiment, the instruction information may include information specifying the user, information on the specimen sampling kit to be provided to the user, and the like.

The specimen sampling kit provider provides the corresponding specimen sampling kit to the user according to the instruction information.

According to an embodiment, all or part of the instruction information may be separately output in the form of an identifier.

According to one embodiment, the provider provides the user with an identifier including all or part of the specimen sampling kit and the instruction information. The identifier may be provided to the user while attached to the specimen sampling kit, or may be provided in a form that the user can attach to the specimen sampling kit.

When the specimen sampling kit includes a plurality of components (e.g., a plurality of containers), an identifier may be provided for each component. The identifiers provided to different components may have information that distinguishes the corresponding component from other components.

The sample sampling kit provider provides a plurality of types of specimen sampling kits, and each specimen sampling kit may include a sampling kit identifier for distinguishing which specimen the corresponding kit is used for sampling. The provider may provide an accurate specimen sampling kit to the user through mutual comparison of the sampling kit identifier and the identifier including all or part of the instruction information.

The specimen sampling kit can be delivered to the user by the provider carrying it to the user's location. Alternatively, the specimen sampling kit can be picked up by the user by moving to the provider's location.

According to one embodiment, when the specimen sampling kit is provided from the provider to the user, there may be included the step of checking whether a specimen sampling kit corresponding to the requesting user is provided.

Whether the user is the requested user may be confirmed through a document that may confirm the user's identity. Alternatively, when the disease test request is made through the user's mobile terminal, an identifier for confirming the user's identity may be generated in the user's mobile terminal, and the user's identity may be confirmed through the identifier. Such confirmation information may be input to the mobile terminal of the provider or the mobile terminal of the user and transmitted to the server.

Whether the provided specimen sampling kit is for the requested user or the corresponding specimen sampling kit is checked using the identifier included in the specimen sampling kit.

In one embodiment, if the user who receives the specimen sampling kit and the user identified through the identifier of the specimen sampling kit are the same and whether the corresponding specimen sampling kit is provided is authenticated, the provider provides the specimen sampling kit to the user.

The provider's provision of the specimen sampling kit to the user may mean not only transfer of occupancy, but also transfer of de facto or possession.

In one embodiment, when sampling a user's specimen, the user may sample the specimen by himself/herself. In particular, in the case of oral wash-out product, saliva, sputum, urine or feces, the user may self-sample the specimen.

In one embodiment, when sampling a user's specimen, a specimen sampling kit provider may collect the specimen from the user. For example, if the specimen sampling kit includes a swab rod, a provider may use the swab rod to collect a specimen from the required area, such as the nose, the throat, and the vagina.

According to an embodiment, when the specimen is collected, the sampling completion information may be provided to the server through the provider's mobile terminal or the user's mobile terminal.

The specimen composition including the collected specimen is stored in a container or bag and transferred to a test facility. An additional sealed container or bag may be included in the specimen sampling kit for sealing the specimen composition and/or the container used for collecting the specimen.

According to one embodiment, the specimen composition may be stored and transported in a frozen state or refrigerated condition.

The container including the specimen composition may contain an identifier assigned to the specimen sampling kit. The test agency can read the identifier to determine whether the user is the subject of the test and the specimen sampling kit.

According to an embodiment, when authentication is performed by comparing the identifier information recognized from the terminal of the test agency with the disease test request information and/or the instruction information of the server and authentication is performed, the test is performed.

The test results of the test agency may be provided to the user or a third party.

According to an embodiment, the method of testing the disease through the specimen sampling of the present invention may be provided through an app, and may be performed by installing the app on a user's mobile terminal, particularly a mobile phone.

According to one embodiment, the method of testing a disease through the specimen sampling of the present invention may be performed by accessing a web server using a computer such as a laptop or a PC.

It may be expressed as a system implementing the method, a program, a storage medium including the program, or the like.

An embodiment of the disease testing method through the specimen sampling of the present invention described above is as follows:

A disease testing method through remote specimen sampling comprises: receiving, by the server, disease test request information provided from the user's mobile terminal wherein the server selects a sampling kit for a disease test corresponding to the disease test request information; providing instruction information to a delivery agency terminal providing the selected sampling kit wherein the instruction information comprises information on a sampling kit for the disease test and delivery information; receiving identifier information from the mobile terminal and authenticating the user using the delivered sampling kit wherein the identifier information is generated by the mobile terminal recognizing an identifier comprised in the sampling kit, and the server checks the identifier information to authenticate the user; authenticating the collected sampling kit by receiving identifier information from a terminal of a test agency wherein the test agency is an agency in which a sampling kit containing the user's specimen is collected, and the identifier information is generated by the terminal of the test agency recognizing an identifier comprised in the sampling kit collected from the user, and the server checks the received identification information to authenticate the sampling kit; receiving a test result from the test agency terminal wherein the test result is generated by a test apparatus performing the disease test on the specimen comprised in the sampling kit; and providing the received test result to the mobile terminal.

Among the matters described in the present specification, matters applicable to the aspects of the present invention have been omitted in order to avoid redundant descriptions, and the above-described matters may be applied to the implementation of other aspects. In particular, matters related to the specimen sampling kits, matters to the providers and the test agency may be mutually applied.

Among the various aspects proposed in the present invention, a place where a user's specimen may be collected using the provided sampling kit may be anywhere the user is located. In particular, there have opened a place where the specimen may be collected under the name of ‘screening test center’ in multi-user facilities such as airports, train stations, subway stations, stadiums, large churches, and government buildings, thereby inducing the users who show suspected symptoms to participate in the test voluntarily.

Other aspects of the present invention can be described as follows.

A central management server may perform a disease testing through remote specimen sampling using a memory, a processor, and one or more programs stored in the memory and be configured to be executed by the processor.

When the one or more programs are executed by one or more processors, it comprises instructions to perform: receiving, by the server, disease test request information provided from the user's mobile terminal wherein the server selects a sampling kit for a disease test corresponding to the disease test request information; providing instruction information to a delivery agency terminal providing the selected sampling kit wherein the instruction information comprises information on a sampling kit for the disease test and delivery information; receiving identifier information from the mobile terminal and authenticating the user using the delivered sampling kit wherein the identifier information is generated by the mobile terminal recognizing an identifier comprised in the sampling kit, and the server checks the identifier information to authenticate the user; authenticating the collected sampling kit by receiving identifier information from a terminal of a test agency wherein the test agency is an agency in which a sampling kit containing the user's specimen is collected, and the identifier information is generated by the terminal of the test agency recognizing an identifier comprised in the sampling kit collected from the user, and the server checks the received identification information to authenticate the sampling kit; receiving a test result from the test agency terminal wherein the test result is generated by a test apparatus performing the disease test on the specimen comprised in the sampling kit; and providing the received test result to the mobile terminal.

A non-transitory computer-readable storage medium that, when executed by one or more processors, a method for managing respiratory infections stores an instruction executed by the one or more processors in a computing device, wherein

A non-transitory computer-readable storage medium that stores an instruction which performs a method for managing respiratory infections by one or more processors in a computing device when executed by the one or more processors.

When the instruction is executed by the one or more processors, the instruction enables the computing device to perform: receiving, by the server, disease test request information provided from the user's mobile terminal wherein the server selects a sampling kit for a disease test corresponding to the disease test request information; providing instruction information to a delivery agency terminal providing the selected sampling kit wherein the instruction information comprises information on a sampling kit for the disease test and delivery information; receiving identifier information from the mobile terminal and authenticating the user using the delivered sampling kit wherein the identifier information is generated by the mobile terminal recognizing an identifier comprised in the sampling kit, and the server checks the identifier information to authenticate the user; authenticating the collected sampling kit by receiving identifier information from a terminal of a test agency wherein the test agency is an agency in which a sampling kit containing the user's specimen is collected, and the identifier information is generated by the terminal of the test agency recognizing an identifier comprised in the sampling kit collected from the user, and the server checks the received identification information to authenticate the sampling kit; receiving a test result from the test agency terminal wherein the test result is generated by a test apparatus performing the disease test on the specimen comprised in the sampling kit; and providing the received test result to the mobile terminal.

Another aspect of the present invention may be described as follows.

A terminal is connected to the above-described server through a communication network and installed with an application that performs the function of a mobile terminal in each step of performing a disease testing method through remote specimen sampling.

Examples described herein relate to methods of managing the respiratory infection or the use of disease test servers via remote specimen sampling to implement the techniques described herein. According to one embodiment, this technique is performed by the central management server or the disease test server in response to a processor executing one or more sequences of one or more instructions included in memory. Such instructions may be read into memory from other machine-readable media such as storage media. Execution of the sequence of instructions included in the memory causes the processor to perform the process steps described herein. In an alternative implementation, hardwired circuitry may be used in place of or in conjunction with software instructions to implement the embodiments described herein. Thus, the described embodiments are not limited to any particular combination of hardware circuitry and software.

One or more examples described herein provide that the methods, techniques, and operations performed by a computing device are performed in a programming scheme or as a computer-implemented method. As used herein, program means use of code or computer-executable instructions. The instructions may be stored in one or more memory resources of a computing device. The steps performed in a programming scheme may, or may not, be automatically performed.

One or more embodiments described herein may be implemented using a server, a unit, or a module. The server, unit, or module may include a software or hardware component capable of performing part of a program or one or more tasks or functions mentioned herein. As used herein, the unit or module may be present on a hardware component independently from other unit or module. Alternatively, the unit or module may be a shared element or process of other unit, module, program, or device.

The examples described herein may be expanded to individual elements and concepts described herein, independently from other concepts, ideas, or systems and may be combined with elements cited anywhere in the present invention. Although some examples have been described in detail with reference to the accompanying drawings, the concept is not limited to such examples. Thus, the scope of the concept is intended to be defined by the appended claims and their equivalents. Further, specific features described individually or as some examples may be combined with other features described individually or other examples although not specifically mentioned for the specific features. Thus, the absence of a description of such combination should not be interpreted as excluding such combination from the scope of the present invention.

### CROSS-REFERENCE TO RELATED APPLICATIONS

This application claims priority from Korean Patent Application No. 10-2020-0035933, filed on Mar. 24, 2020 and Korean Patent Application No. 10-2020-0038634 filed on Mar. 30, 2020, in the Korean Intellectual Property Office, the disclosure of which is incorporated herein by reference in its entirety.

1. A method comprising the following steps of managing respiratory infections through a mobile terminal management system comprising a central management server,
receiving, by the central management server, a request for a respiratory infection test from a client's mobile terminal;
receiving, by the central management server, location information from the mobile terminal to generate movement information;
requesting, by the central management server, the respiratory infection test for the client to a terminal of a test agency;
receiving, by the central management server, a test result of the respiratory infection of the client from the terminal of the test agency; and
generating, by the central management server, the respiratory infection management information using the movement information and the test result.

2. The method of claim 1, further comprising providing the respiratory infection management information to a mobile terminal of at least one other client.

3. The method of claim 1, wherein the respiratory infection management information is provided to the mobile terminal.

4. The method of claim 1, wherein the respiratory infection management information comprises the movement information of at least one of the plurality of clients who applied for the respiratory infection test.

5. The method of claim 4, wherein the respiratory infection management information is displayed on a digital map.

6. The method of claim 1, wherein the location information is real-time location information of the mobile terminal.

7. The method of claim 1, wherein the respiratory infection test request comprises information input through an application installed in the mobile terminal.

8. The method of claim 7, wherein the information comprises questionnaire information on the respiratory infection.

9. The method of claim 1, further comprising, when the test result of the respiratory infection is determined as positive,
requesting, by the central management server, a treatment for the respiratory infection of the client to a terminal of a treatment agency; and
receiving, by the central management server, treatment information for the treatment from the terminal of the treatment agency.

10. A central management server for managing respiratory infections using a memory, a processor, and one or more programs stored in the memory and configured to be executed by the processor, wherein
when the one or more programs are executed by one or more processors, the one or more programs comprises instructions to perform a method comprising the following step:
receiving, by the central management server, a request for a respiratory infection test from a client's mobile terminal;
receiving, by the central management server, location information from the mobile terminal to generate movement information;
requesting, by the central management server, the respiratory infection test for the client to a terminal of a test agency;
receiving, by the central management server, a test result of the respiratory infection of the client from the terminal of the test agency; and
generating, by the central management server, the respiratory infection management information using the movement information and the test result.

11. A non-transitory computer-readable storage medium that stores an instruction which performs a method for managing respiratory infections by one or more processors in a computing device when executed by the one or more processors, wherein
when the instruction is executed by the one or more processors, the instruction enables the computing device to perform a method comprising the following step:
receiving, by the central management server, a request for a respiratory infection test from a client's mobile terminal;
receiving, by the central management server, location information from the mobile terminal to generate movement information;
requesting, by the central management server, the respiratory infection test for the client to a terminal of a test agency;
receiving, by the central management server, a test result of the respiratory infection of the client from the terminal of the test agency; and
generating, by the central management server, the respiratory infection management information using the movement information and the test result.

12. A terminal connected to the central management server of claim 1 through a communication network and installed with an application that performs the function of a mobile terminal in each step of performing a method of managing respiratory infections.

13.-26. (canceled)
